(19) United States
(12) Patent Application Publication
(10) Pub. No.: US 2023/0245735 A1
(43) Pub. Date: Aug. 3, 2023

(54) ELECTRONIC MEDICAL RECORD DATA ANALYSIS SYSTEM AND ELECTRONIC MEDICAL RECORD DATA ANALYSIS METHOD

(71) Applicant: ASUSTeK COMPUTER INC., Taipei City (TW)

(72) Inventors: Tsung-Han Yang, Taipei City (TW); Chia-Wen Tsai, Taipei City (TW); Jie-Jyun Liu, Taipei City (TW); Ting-Hsuan Wang, Taipei City (TW); Cheng Jeng, Taipei City (TW); Wen-Han Wu, Taipei City (TW)

(73) Assignee: ASUSTeK COMPUTER INC., Taipei City (TW)

(21) Appl. No.: 17/851,069

(22) Filed: Jun. 28, 2022

(30) Foreign Application Priority Data
Jan. 28, 2022 (TW) 111103810

(51) Int. Cl. G16H 10/60; G16H 50/20; G06F 40/10

(52) U.S. Cl. CPC G16H 10/60; G16H 50/20; G06F 40/10

(57) ABSTRACT

An electronic medical record data analysis system and an electronic medical record data analysis method are provided. The electronic medical record data analysis system includes a storage device and a processor. The storage device is configured to store an electronic medical record data analysis module and a post-processing module. The processor obtains electronic medical record data. The processor executes the electronic medical record data analysis module to analyze the electronic medical record data and generate a plurality of disease diagnosis codes and a plurality of correlation degree scores corresponding to the electronic medical record data. The processor sorts the plurality of disease diagnosis codes according to the plurality of correlation degree scores, to generate an initial list, and executes the post-processing module to post-process the initial list according to a preset coding rule. The processor generates a recommendation list according to the post-processed initial list.

### CROSS-REFERENCE TO RELATED APPLICATION

This application claims the priority benefit of Taiwan Application Serial No. 111103810, filed on Jan. 28, 2022. The entirety of the above-mentioned patent application is hereby incorporated by reference herein and made a part of the specification.

### BACKGROUND OF THE INVENTION

### Field of the Invention

This disclosure relates to an electronic medical record data analysis system and an electronic medical record data analysis method.

### Description of the Related Art

Generally, in a diagnosis process of a patient, medical personnel establish electronic medical record data for relevant diagnosis analysis and recording. In this regard, the medical personnel need to manually determine the current electronic medical record data, to generate corresponding International Classification of Diseases (ICD) codes. Therefore, traditional analysis and filing operations of the electronic medical record data are inefficient and time-consuming. In addition, as the version of the ICD codes is updated, the quantity of codes increases and the coding rule becomes more complex. As a result, the medical personnel need to spend more time and energy on the analysis and filing operations of the electronic medical record data.

### BRIEF SUMMARY OF THE INVENTION

According to the first aspect, an electronic medical record data analysis system is provided. The electronic medical record data analysis system includes a storage device and a processor. The storage device is configured to store an electronic medical record data analysis module and a post-processing module. The processor is coupled to the storage device, and obtains electronic medical record data. The processor executes the electronic medical record data analysis module to analyze the electronic medical record data and generate a plurality of disease diagnosis codes and a plurality of correlation degree scores corresponding to the electronic medical record data. The processor sorts the plurality of disease diagnosis codes according to the plurality of correlation degree scores, to generate an initial list, and the processor executes the post-processing module to post-process the initial list according to a preset coding rule. The processor generates a recommendation list according to the post-processed initial list.

According to the second aspect, an electronic medical record data analysis method is provided. The electronic medical record data analysis method include the following steps: obtaining electronic medical record data; executing an electronic medical record data analysis module to analyze the electronic medical record data and generate a plurality of disease diagnosis codes and a plurality of correlation degree scores corresponding to the electronic medical record data; sorting the plurality of disease diagnosis codes according to the plurality of correlation degree scores, to generate an initial list; executing a post-processing module to post-process the initial list according to a preset coding rule; and generating a recommendation list according to the post-processed initial list.

Based on the above, according to the electronic medical record data analysis system and the electronic medical record data analysis method of this disclosure, a corresponding recommendation list of disease diagnosis codes is automatically generated according to an analysis result of inputted electronic medical record data, to implement a convenient and reliable medical diagnosis auxiliary function.

To make the foregoing features and advantages of this disclosure clear and easy to understand, the following gives a detailed description of embodiments with reference to accompanying drawings.

### BRIEF DESCRIPTION OF THE DRAWINGS

FIG. 1 is a schematic diagram of an electronic medical record data analysis system according to an embodiment of this disclosure.

FIG. 2 is a flowchart of an electronic medical record data analysis method according to an embodiment of this disclosure.

FIG. 3 is a schematic analysis diagram of electronic medical record data according to an embodiment of this disclosure.

FIG. 4 is a schematic implementation diagram of an attention mechanism according to an embodiment of this disclosure.

FIG. 5 is a flowchart of model training according to an embodiment of this disclosure.

### DETAILED DESCRIPTION OF THE EMBODIMENTS

To make the content of this disclosure more comprehensible, the embodiments are described below as examples according to which this disclosure can indeed be implemented. In addition, wherever possible, elements/components/steps with same reference numerals in the drawings and implementations represent same or similar parts.

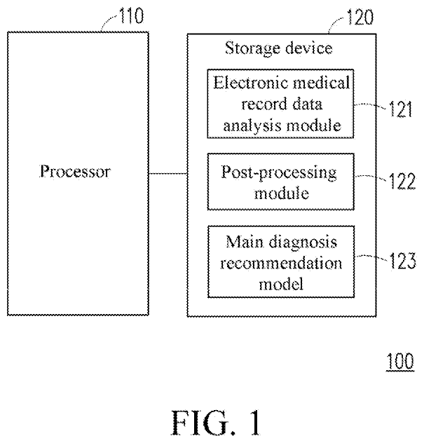

Referring to FIG. 1, an electronic medical record data analysis system 100 includes a processor 110 and a storage device 120. The processor 110 is coupled to the storage device 120. In this embodiment, the storage device 120 stores an electronic medical record data analysis module 121, a post-processing module 122, and a main diagnosis recommendation model 123. The electronic medical record data analysis module 121, the post-processing module 122, and the main diagnosis recommendation model 123 are integrated into an artificial intelligence (AI) model. The processor 110 executes the electronic medical record data analysis module 121 to analyze electronic medical record data and automatically generate a plurality of corresponding disease diagnosis codes and a plurality of corresponding correlation degree scores. The processor 110 arranges the plurality of disease diagnosis codes according to the plurality of disease diagnosis codes and the plurality of correlation degree scores, to generate a list, and adjusts the list by executing the post-processing module 122 and the main diagnosis recommendation model 123, to generate a final recommendation list including the plurality of disease diagnosis codes.

In this embodiment, the electronic medical record data, in an embodiment, includes text information such as a current admission diagnosis, and a subjective complaint, and/or a diagnosis of a patient. In this embodiment, the plurality of disease diagnosis codes is International Classification of Diseases 10th Revision (ICD-10) codes.

In this embodiment, the processor 110 is, in an embodiment, a central processing unit (CPU) including data processing and computing functions, or a microprocessor including other programmable general purposes or special purposes, a digital signal processor (DSP), an image processing unit (IPU), a graphics processing unit (GPU), a programmable controller, an application specific integrated circuit (ASIC), a programmable logic device (PLD) or other similar processing devices, or a combination of thereof. The storage device 120 includes, but is not limited to, a memory, in an embodiment, a non-volatile memory (NVM), and stores a plurality of models, modules, programs, and/or algorithms to analyze the electronic medical record data of this disclosure.

In this embodiment, the electronic medical record data analysis system 100 is implemented by, in an embodiment, being integrated in a desktop computer, a personal computer (PC), or a tablet computer (Tablet PC). In an embodiment, the storage device 120 is, in an embodiment, set in a cloud server, and related models and modules stored in the storage device 120 are executed by the processor 110 of a computer device operated by medical personnel. In addition, the electronic medical record data analysis system 100 further includes an input device and a communication device. The input device is configured to receive electronic medical record data inputted by the medical personnel, and the communication device is configured to be connected to a medical record database, so that the electronic medical record data analysis system 100 obtains historical electronic medical record data to train the related models and modules.

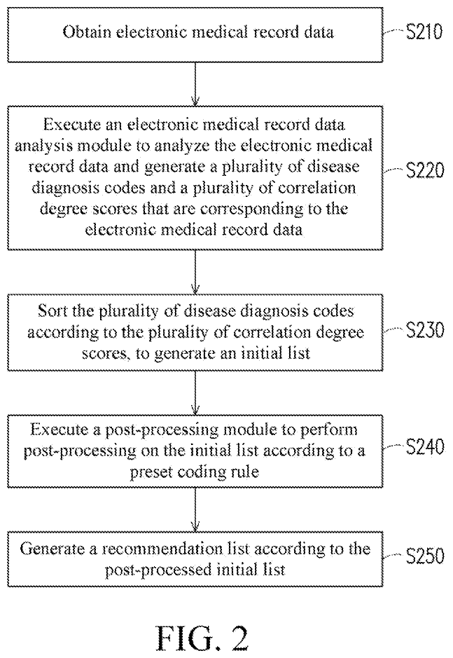

Referring to FIG. 1 and FIG. 2, in an embodiment, the electronic medical record data analysis system 100 executes the following steps S210 to S250. In this embodiment, the medical personnel input current electronic medical record data of a patient into the electronic medical record data analysis system 100. In step S210, the processor 110 obtains electronic medical record data. In step S220, the processor 110 executes the electronic medical record data analysis module 121 to analyze the electronic medical record data and generate a plurality of disease diagnosis codes and a plurality of correlation degree scores corresponding to the electronic medical record data. In this embodiment, the plurality of correlation degree scores respectively represents correlation degrees (or confidence values) between the plurality of disease diagnosis codes and the electronic medical record data. In step S230, the processor 110 sorts the plurality of disease diagnosis codes according to the plurality of correlation degree scores, to generate an initial list; The processor 110 first sequentially arranges the plurality of disease diagnosis codes with higher correlation degree scores to lower correlation degree scores, to generate the initial list.

In step S240, the processor 110 executes the post-processing module 122 to post-process the initial list according to a preset coding rule. In this embodiment, the preset coding rule, in an embodiment, refers to a specific coding rule of the ICD-10. The processor 110 resorts arrangement sequences of the plurality of disease diagnosis codes in the initial list according to the specific coding rule of the ICD-10. In step S250, the processor 110 generates a recommendation list according to the post-processed initial list. In this embodiment, the processor 110, in an embodiment, adjusts arrangement sequences of the plurality of disease diagnosis codes in the post-processed initial list according to historical medical record data of the patient, to generate a final recommendation list, and the recommendation list is displayed by a display device. In addition, the medical personnel select a disease diagnosis code in the recommendation list by operating the input device, to obtain the most relevant main diagnosis information of the current medical treatment of the patient.

Therefore, according to the electronic medical record data analysis system 100 and the electronic medical record data analysis method of this disclosure, the electronic medical record data of the patient who is currently intended to undergo a medical diagnosis is automatically analyzed, to instantly generate a corresponding disease diagnosis code, thereby implementing a convenient medical diagnosis auxiliary function.

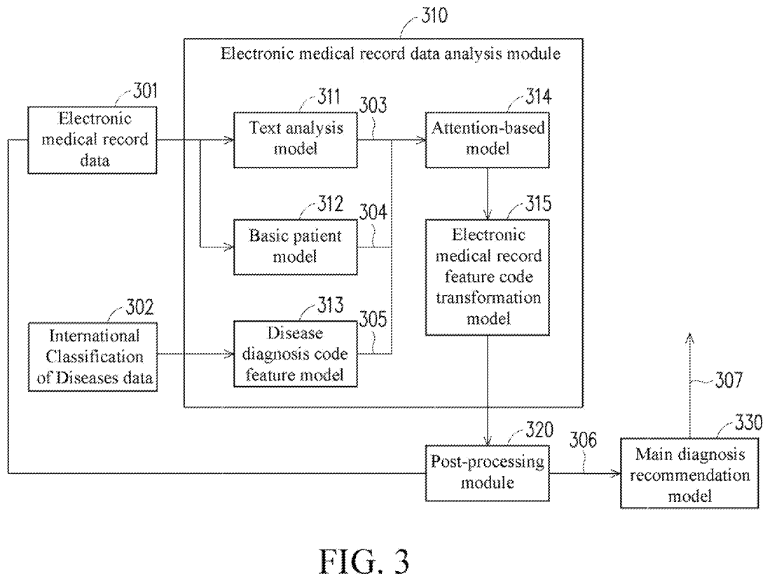

Referring to FIG. 3, this embodiment further describes an analysis process of electronic medical record data. In this embodiment, a processor of an electronic medical record data analysis system (in an embodiment, the processor 110 of the electronic medical record data analysis system 100 in FIG. 1) executes an electronic medical record data analysis module 310, a post-processing module 320, and a main diagnosis recommendation model 330, and obtains electronic medical record data 301 and International Classification of Disease data 302. The International Classification of Disease data 302 is, in an embodiment, relevant disease diagnosis texts of ICD-10 codes.

In this embodiment, the electronic medical record data analysis module 310 includes a text analysis model 311, a basic patient model 312, a disease diagnosis code feature model 313, an attention-based model 314, and an electronic medical record feature code transformation model 315. In this embodiment, the text analysis model 311 first performs natural language processing (NLP) on the electronic medical record data 301 to identify semantics of words, texts, and/or tokens in a medical record text field of the electronic medical record data 301. In this embodiment, the text analysis model 311 generates a plurality of medical record feature parameters 303 and provides the plurality of medical record feature parameters 303 to the attention-based model 314. In addition, the text analysis model 311 of this embodiment is further implemented by matching a long-document transformer (longformer), to effectively increase a text length to be processed by the text analysis model 311.

In this embodiment, the basic patient model 312 analyzes the electronic medical record data 301 to determine a relevant basic medical term. The basic patient model 312 generates a plurality of basic patient feature parameters 304 and provides the plurality of basic patient feature parameters to the attention-based model 314. In this embodiment, the disease diagnosis code feature model 313 analyzes the International Classification of Diseases data 302. The disease diagnosis code feature model 313 generates a plurality of diagnosis code feature parameters 305 (a disease diagnosis code, in an embodiment, corresponding to a plurality of feature parameters), and provides the plurality of diagnosis code feature parameters to the attention-based model 314. In this embodiment, the attention-based model 314 highlights the plurality of disease diagnosis codes at a plurality of corresponding positions in the electronic medical record data 301 respectively according to the plurality of medical record feature parameters 303, the plurality of diagnosis code feature parameters 305, and the plurality of basic patient feature parameters 304. In this embodiment, the attention-based model 314 compares a similarity between the plurality of medical record feature parameters 303 and the plurality of diagnosis code feature parameters 305, and compares a similarity between the plurality of medical record feature parameters 303 and the plurality of basic patient feature parameters 304. It is to be noted that the electronic medical record data analysis system further includes a display device. The electronic medical record data analysis system displays the electronic medical record data 301 through the display device, and uses a label embedding method and a document embedding method to highlight a plurality of texts or tokens corresponding to the plurality of medical record feature parameters 303 in the electronic medical record data 301, so that the medical personnel intuitively focus on the highlighted keywords, texts, or tokens in the electronic medical record data 301 through the display device.

In this embodiment, the electronic medical record feature code transformation model 315 calculates a plurality of correlation degree scores corresponding to the plurality of disease diagnosis codes according to a determining result of the attention-based model 314. The electronic medical record data analysis system performs sorting according to the plurality of disease diagnosis codes and the plurality of correlation degree scores, to generate an initial list. In this embodiment, the post-processing module 320 rearranges the initial list according to a preset coding rule (a specific coding rule of the ICD-10) and patient information in the electronic medical record data 301. In this embodiment, the main diagnosis recommendation model 330, in an embodiment, adjusts arrangement sequences of the plurality of disease diagnosis codes in the post-processed initial list 306 according to historical medical record data of the patient, to generate a recommendation list 307. In this way, the medical personnel, in an embodiment, select a disease diagnosis code in the recommendation list displayed by the display device by operating the input device, so that the processor of the electronic medical record data analysis system immediately reads main diagnosis information corresponding to the disease diagnosis code, to immediately obtain the most relevant main diagnosis information of the current medical treatment of the patient.

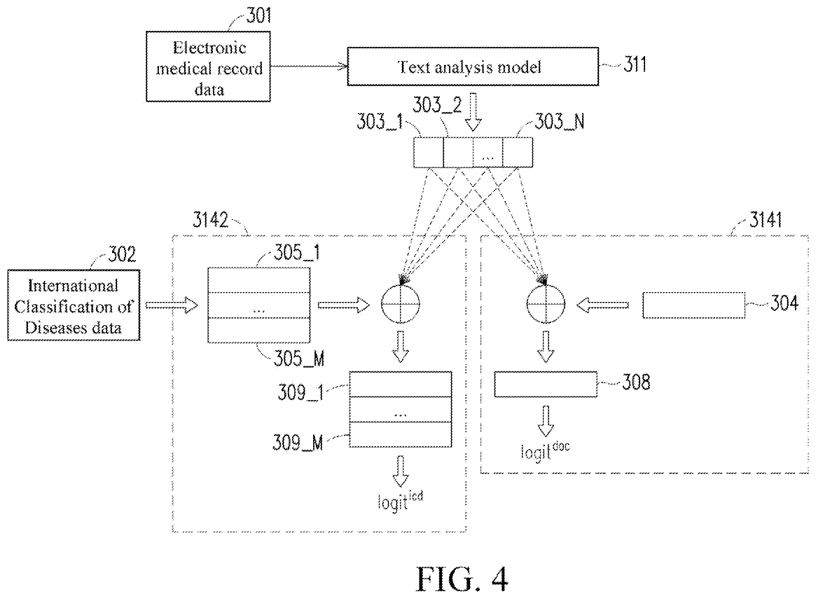

FIG. 4 is a schematic implementation diagram of an attention mechanism according to an embodiment of this disclosure. Referring to FIG. 3 and FIG. 4, this embodiment further describes the implementation of the attention mechanism. In this embodiment, the processor of the electronic medical record data analysis system (in an embodiment, the processor 110 of the electronic medical record data analysis system 100 in FIG. 1) inputs the electronic medical record data 301 into the text analysis model 311, so that the text analysis model 311 generates a plurality of medical record feature parameters 303_1 to 303_N, where N is a positive integer. The medical record feature parameters 303_1 to 303_N are, in an embodiment, features of a plurality of words, texts, and/or tokens in the electronic medical record data 301.

In this embodiment, the attention-based model 314 includes a patient representation model 3141 (label-wise document attention layer) and a label representation model 3142 (document attention layer). The patient representation model 3141 compares a similarity between the medical record feature parameters 303_1 to 303_N and the plurality of basic patient feature parameters 304, to generate a plurality of first assessment features 308 (or referred to as case assessment features). The label representation model 3142 compares a similarity between the medical record feature parameters 303_1 to 303_N and a plurality of diagnosis code feature parameters 305_1 to 305_M that is corresponding to different diagnosis codes and is generated based on the International Classification of Diseases data 302, to generate a plurality of second assessment features 309_1 to 309_M (or referred to as a plurality of disease diagnosis code assessment features), where M is a positive integer.

In this embodiment, the electronic medical record feature code transformation model 315 calculates a plurality of correlation degree scores corresponding to the plurality of disease diagnosis codes according to the plurality of first assessment features 308 and the plurality of second assessment features 309_1 to 309_M. In this case, the electronic medical record feature code transformation model, in an embodiment, executes the following formula (1) to perform a sigmoid formula operation on a logit^{icd} function corresponding to the plurality of first assessment features 308 and a logit^{doc} function corresponding to the plurality of second assessment features 309_1 to 309_M, to obtain a correlation degree score ŷ or referred to as a confidence value, which is expressed as a percentage) between 0 and 1.

$$\hat{y} = sigmoid ( \frac{{logit}^{doc} + {logit}^{icd}}{2} ) \qquad formula ( 1 )$$

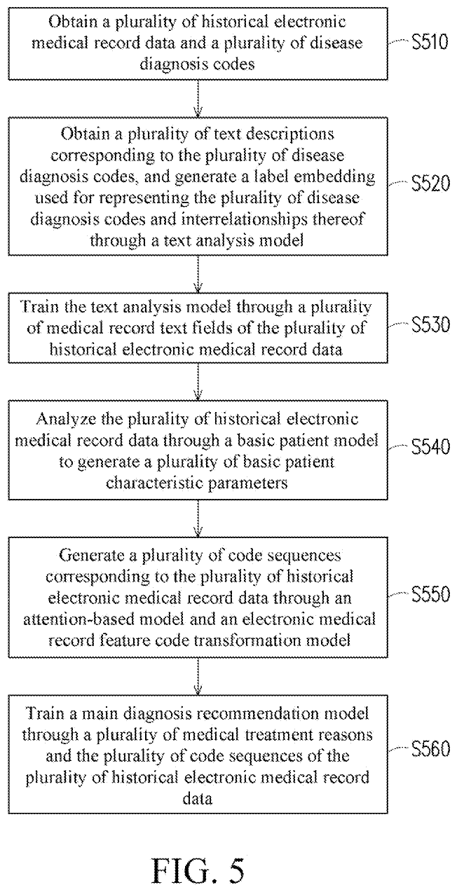

FIG. 5 is a flowchart of model training according to an embodiment of this disclosure. Referring to FIG. 3 and FIG. 5, the electronic medical record data analysis system performs the following steps S510 to S560 in advance, to train models. In step S510, the electronic medical record data analysis system obtains a plurality of pieces of historical electronic medical record data and a plurality of disease diagnosis codes. In this embodiment, the electronic medical record data analysis system is connected to a medical record database, to obtain the plurality of historical electronic medical record data and the plurality of corresponding disease diagnosis codes. The plurality of historical electronic medical record data includes, in an embodiment, admission and discharge diagnosis data, surgical record data, SOAP (subjective, objective, assessment, and plan) data, medical history data, and disease course data.

In step S520, the electronic medical record data analysis system obtains a plurality of text descriptions corresponding to the plurality of disease diagnosis codes, and generates a plurality of label embeddings used for representing the plurality of disease diagnosis codes and interrelationships thereof through the text analysis model 311. In this embodiment, the electronic medical record data analysis system obtains, in an embodiment, all disease diagnosis codes of the ICD-10 and relevant disease diagnosis descriptions, and performs semantic identification through the text analysis model 311, to generate a plurality of label embeddings used for representing the plurality of disease diagnosis codes and interrelationships thereof.

In step S530, the electronic medical record data analysis system trains the text analysis model 311 through the plurality of medical record text fields of the plurality of pieces of historical electronic medical record data. In this embodiment, the electronic medical record data analysis system trains the text analysis model 311 based on bidirectional encoder representations from transformers (BERT) with the medical field as a main task, and a knowledge distillation technology of machine learning is used to learn medical knowledge through a smaller BERT model, so that the text analysis model 311 reduces system requirements, speeds up operations, and achieve better generalized text understanding capabilities.

In step S540, the electronic medical record data analysis system analyzes the plurality of pieces of historical electronic medical record data through the basic patient model 312, to generate a plurality of basic patient feature parameters. In step S550, the electronic medical record data analysis system generates a plurality of code sequences corresponding to the plurality of pieces of historical electronic medical record data through the attention-based model 314 and the electronic medical record feature code transformation model 315. In this embodiment, the attention-based model 314 and the electronic medical record feature code transformation model 315 perform the relevant operation of the attention mechanism as described in the foregoing embodiment of FIG. 4 according to the foregoing obtained feature parameters, to generate the plurality of code sequences corresponding to the plurality of pieces of historical electronic medical record data.

In step S560, the electronic medical record data analysis system trains the main diagnosis recommendation model 330 through a plurality of medical treatment reasons and the plurality of code sequences of the plurality of pieces of historical electronic medical record data. In this way, the trained main diagnosis recommendation model 330 effectively adjusts the arrangement sequences of the plurality of disease diagnosis codes in the post-processed initial list 306, to generate the correct recommendation list 307 corresponding to the electronic medical record data 301 inputted currently by the medical personnel.

In addition, the electronic medical record data analysis system of this disclosure further updates and optimizes the foregoing modules and models according to user feedback loops, to continuously train modules and models that are more suitable for user experience. In an embodiment, the electronic medical record data analysis system uses the electronic medical record data, the analysis result, and the main diagnosis selection result that are inputted by the medical personnel each time to update the historical electronic medical record data (as new training data), to continuously train the foregoing modules and models.

To sum up, according to the electronic medical record data analysis system and the electronic medical record data analysis method of this disclosure, a corresponding recommendation list of disease diagnosis codes is automatically generated according to an analysis result of inputted electronic medical record data and the disease diagnosis codes are highlighted on the electronic medical record data. In this way, the medical personnel instantly and intuitively obtain main diagnosis information and key medical record information of the current diagnosis of the patient through the recommendation list displayed by the display device and the highlighted electronic medical record data.

Although this disclosure has been described with reference to the above embodiments, the embodiments are not intended to limit this disclosure. A person of ordinary skill in the art may make variations and improvements without departing from the spirit and scope of this disclosure. Therefore, the protection scope of this disclosure should be subject to the appended claims.

What is claimed is:

1. An electronic medical record data analysis system, comprising:
a storage device, configured to store an electronic medical record data analysis module and a post-processing module; and
a processor, coupled to the storage device, and configured to obtain electronic medical record data, wherein the processor executes the electronic medical record data analysis module to analyze the electronic medical record data and generate a plurality of disease diagnosis codes and a plurality of correlation degree scores corresponding to the electronic medical record data;
the processor sorts the plurality of disease diagnosis codes according to the plurality of correlation degree scores, to generate an initial list, and the processor executes the post-processing module to post-process the initial list according to a preset coding rule; and the processor generates a recommendation list according to the post-processed initial list.

2. The electronic medical record data analysis system according to claim 1, wherein the electronic medical record data analysis module comprises:
a text analysis model, configured to analyze the electronic medical record data to generate a plurality of medical record feature parameters;
a disease diagnosis code feature model, configured to analyze International Classification of Diseases data to generate a plurality of diagnosis code feature parameters;
a basic patient model, configured to analyze the electronic medical record data to generate a plurality of basic patient feature parameters; and
an attention-based model, configured to highlight the plurality of disease diagnosis codes at a plurality of corresponding positions in the electronic medical record data respectively according to the plurality of medical record feature parameters, the plurality of diagnosis code feature parameters, and the plurality of basic patient feature parameters.

3. The electronic medical record data analysis system according to claim 2, wherein the attention-based model is further configured to compare a similarity between the plurality of medical record feature parameters and the plurality of basic patient feature parameters, to generate a plurality of first assessment features, and compare a similarity between the plurality of medical record feature parameters and the plurality of diagnosis code feature parameters, to generate a plurality of second assessment features; and the electronic medical record data analysis module further comprises:
an electronic medical record feature code transformation model, configured to generate a plurality of first assessment scores according to the plurality of first assessment features, generate a plurality of second assessment scores according to the plurality of second assessment features, and calculate the plurality of correlation degree scores corresponding to the plurality of disease diagnosis codes according to the plurality of first assessment scores and the plurality of second assessment scores.

4. The electronic medical record data analysis system according to claim 2, wherein the processor trains the text analysis model in advance through a plurality of medical record text fields of a plurality of pieces of historical electronic medical record data.

5. The electronic medical record data analysis system according to claim 4, wherein the text analysis model comprises a long-document transformer (longformer).

6. The electronic medical record data analysis system according to claim 4, wherein the electronic medical record data analysis module further comprises:
a main diagnosis recommendation model, configured to generate the recommendation list according to the post-processed initial list.

7. The electronic medical record data analysis system according to claim 6, wherein the processor trains the main diagnosis recommendation model in advance through an individual medical treatment reason and a code sequence of the plurality of pieces of historical electronic medical record data.

8. The electronic medical record data analysis system according to claim 7, wherein the processor updates the electronic medical record data and the plurality of disease diagnosis codes into the plurality of pieces of historical electronic medical record data.

9. The electronic medical record data analysis system according to claim 1, wherein the processor selects one of the plurality of disease diagnosis codes from the recommendation list according to a selection instruction, and obtains corresponding main diagnosis information according to the one of the plurality of disease diagnosis codes.

10. The electronic medical record data analysis system according to claim 1, wherein the plurality of disease diagnosis codes is International Classification of Diseases 10th Revision (ICD-10) codes.

11. An electronic medical record data analysis method, comprising:
obtaining electronic medical record data;
executing an electronic medical record data analysis module to analyze the electronic medical record data and generate a plurality of disease diagnosis codes and a plurality of correlation degree scores corresponding to the electronic medical record data;
sorting the plurality of disease diagnosis codes according to the plurality of correlation degree scores, to generate an initial list;
executing a post-processing module to post-process the initial list according to a preset coding rule; and
generating a recommendation list according to the post-processed initial list.

12. The electronic medical record data analysis method according to claim 11, wherein the step of executing the electronic medical record data analysis module to analyze the electronic medical record data comprises:
analyzing the electronic medical record data through a text analysis model, to generate a plurality of medical record feature parameters;
analyzing International Classification of Diseases data through a disease diagnosis code feature model, to generate a plurality of diagnosis code feature parameters;
analyzing the electronic medical record data through a basic patient model, to generate a plurality of basic patient feature parameters; and
highlighting the plurality of disease diagnosis codes at a plurality of corresponding positions in the electronic medical record data respectively through an attention-based model according to the plurality of medical record feature parameters, the plurality of diagnosis code feature parameters, and the plurality of basic patient feature parameters.

13. The electronic medical record data analysis method according to claim 12, wherein the step of executing the electronic medical record data analysis module to analyze the electronic medical record data further comprises:
comparing a similarity between the plurality of medical record feature parameters and the plurality of basic patient feature parameters through the attention-based model, to generate a plurality of first assessment features, and comparing a similarity between the plurality of medical record feature parameters and the plurality of diagnosis code feature parameters, to generate a plurality of second assessment features; and
generating a plurality of first assessment scores through an electronic medical record feature code transformation model according to the plurality of first assessment features, generating a plurality of second assessment scores according to the plurality of second assessment features, and calculating the plurality of correlation degree scores corresponding to the plurality of disease diagnosis codes according to the plurality of first assessment scores and the plurality of second assessment scores.

14. The electronic medical record data analysis method according to claim 12, further comprising:
training the text analysis model in advance through a plurality of medical record text fields of a plurality of pieces of historical electronic medical record data.

15. The electronic medical record data analysis method according to claim 14, wherein the text analysis model comprises a long-document transformer (longformer).

16. The electronic medical record data analysis method according to claim 14, wherein the step of generating a recommendation list according to the post-processed initial list comprises:
generating the recommendation list through a main diagnosis recommendation model according to the post-processed initial list.

17. The electronic medical record data analysis method according to claim 16, further comprising:
training the main diagnosis recommendation model in advance through a plurality of medical treatment reasons and a plurality of code sequences of the plurality of pieces of historical electronic medical record data.

18. The electronic medical record data analysis method according to claim 17, further comprising:
updating the electronic medical record data and the plurality of disease diagnosis codes into the plurality of pieces of historical electronic medical record data.

19. The electronic medical record data analysis method according to claim 11, further comprising:
selecting one of the plurality of disease diagnosis codes from the recommendation list according to a selection instruction, and obtaining corresponding main diagnosis information according to the one of the plurality of disease diagnosis codes.

20. The electronic medical record data analysis method according to claim 11, wherein the plurality of disease diagnosis codes is International Classification of Diseases 10th Revision (ICD-10) codes.
